(19) United States
(12) Patent Application Publication
(10) Pub. No.: US 2015/0335818 A1
(43) Pub. Date: Nov. 26, 2015

(54) SELF CONTAINED SELF INFLATING REGULATED CONTAINER

(71) Applicant: MEYERS & GERARD LLC, Long Island City, NY (US)

(72) Inventors: Michael B. MEYERS, Sea Cliff, NY (US); Mark P. GERARD, Long Island City, NY (US)

(21) Appl. No.: 14/440,348

(22) PCT Filed: Oct. 21, 2013
(86) PCT No.: PCT/US2013/065811

§ 371 Date: May 1, 2015

Related U.S. Application Data
(63) Continuation-in-part of application No. 13/804,397, filed on Mar. 14, 2013.
(60) Provisional application No. 61/795,566, filed on Oct. 19, 2012.
(60) Provisional application No. 61/795,566, filed on Oct. 19, 2012.

(51) Int. Cl. A61M 5/155; A61M 5/148

(52) U.S. Cl. CPC A61M 5/155; A61M 5/1483; A61M 2205/3331

(57) ABSTRACT

A container has a first compartment, a sleeve configured to receive at least one container for holding fluid, and a second compartment coupled to the first compartment, outside of the first compartment. Disposed inside of the second compartment in a substantially sealed manner is at least one inflator configured to pump a fluid into the first compartment wherein the at least one inflator. There can be at least one fluid channel coupled to the first compartment and to the at least one inflator for allowing fluid to flow from the at least one inflator to the first compartment.

### CROSS REFERENCE TO RELATED APPLICATIONS

This application is a non provisional application that claims priority under 35 U.S.C. 119e from U.S. Provisional application Ser. No. 61/795,566 filed on Oct. 19, 2012, the disclosure of which is hereby incorporated herein by reference in its entirety. This application is also a continuation in part application of U.S. patent application Ser. No. 13/804,397 filed on Mar. 14, 2013 the disclosure of which is hereby incorporated herein by reference in its entirety. The '397 application also claims priority from, and is a non provisional of 61/795,566 filed on Oct. 19, 2012.

### BACKGROUND OF THE INVENTION

At least one embodiment of the invention relates to a self-contained self-inflating infusion bag that can include multiple compartments. In at least one embodiment, the self contained infusion bag can have a bleeder valve and/or all compartments that extend substantially parallel to each other. It has been determined that there is a need for a self contained infusion bag that is simple to operate, is compact and easy to use.

### SUMMARY OF THE INVENTION

At least one embodiment of the invention relates to a container comprising a first compartment, a sleeve configured to receive at least one container for holding fluid, and a second compartment coupled to the first compartment, outside of the first compartment. Disposed inside of the second compartment in a substantially sealed manner is at least one inflator configured to pump a fluid into the first compartment via the inflator. There can be at least one fluid channel coupled to the first compartment and to the at least one inflator for allowing fluid to flow from the at least one inflator to the first compartment.

In at least one embodiment, the first compartment can be a bladder with a substantially finite volume that is substantially sealed such that substantially all of the fluid is expressed into and from the first compartment and expressed through the at least one fluid channel.

Disposed in line along the fluid channel is at least one valve for selectively allowing fluid to flow into and out of the bladder or first compartment. In at least one embodiment, this valve is a three-way valve which allows fluid to flow along the fluid channel. In at least one embodiment, this three-way valve comprises a valve for allowing fluid to pass from the inflator to the at least one compartment and at least one bleeder valve for allowing fluid to be expressed from at least one of the first compartment and the inflator. In at least one embodiment, the three-way valve is configured to allow fluid to be expressed from the first compartment while preventing fluid from being expressed from the inflator.

In at least one embodiment, the inflator comprises a pressurized cartridge. In at least one embodiment, the pressurized cartridge comprises a propellant filled cartridge housing at least one fluid under pressure. In at least one embodiment, the pressurized cartridge comprises a pressurized CO₂ cartridge having at least one section for selectively opening the pressurized cartridge.

In at least one embodiment, the second compartment is open to receive the at least one fluid port but is substantially sealed. In at least one embodiment, the first compartment is open to receive the at least one fluid port but is substantially sealed.

In at least one embodiment, the first compartment extends along a longitudinal axis and the second compartment extends along a longitudinal axis wherein the longitudinal axis of the first compartment is substantially parallel with the longitudinal axis of the second compartment.

In at least one embodiment, the device can further comprise at least one pressure gauge. In at least one embodiment, the pressure gauge is coupled in line with the fluid channel.

In at least one embodiment, the inflator comprises at least one pressurized container, at least one housing, and at least one actuator configured to selectively actuate the inflator. In at least one embodiment, the actuator comprises a drive and at least one opener, wherein the drive is configured to drive the pressurized container into the opener to selectively open the at least one pressurized container. In at least one embodiment, the housing for the inflator comprises at least two parts of a housing made from rigid plastic

In at least one embodiment, a first part of the housing is movable relative to the at least one second part of the housing to adjust a pressure on the at least one pressure regulator.

In at least one embodiment, there is at least one pressure regulator disposed between the at least one first compartment and the at least one second compartment. In at least one embodiment, the pressure regulator is coupled to the least one first part of the housing.

### BRIEF DESCRIPTION OF THE DRAWINGS

Other objects and features of the present invention will become apparent from the following detailed description considered in connection with the accompanying drawings which disclose at least one embodiment of the present invention. It should be understood, however, that the drawings are designed for the purpose of illustration only and not as a definition of the limits of the invention.

In the drawings, wherein similar reference characters denote similar elements throughout the several views:

FIG. 1 is a side view of a first embodiment of the device;

FIG. 2 is a back side view of the container;

FIG. 3 is a side view of the device according to a first embodiment;

FIG .4 is a side view of the pressure regulating section of the invention;

FIG. 5 is side view of another embodiment of the pressure regulating section;

FIG. 6 is a side view of another embodiment of the manifold and pressure regulating section;

FIG. 7 is a side view of the dial control system;

FIG. 8 is another embodiment of the container system;

FIG. 9 is a front view of another embodiment of the container system;

FIG. 10 is a back view of another embodiment of the container system;

FIG. 11 is a side view of the embodiment shown in FIGS. 9 and 10 in the position to expand; and

FIG. 12 is a view of another embodiment of the container system.

### DETAILED DESCRIPTION OF PREFERRED EMBODIMENTS

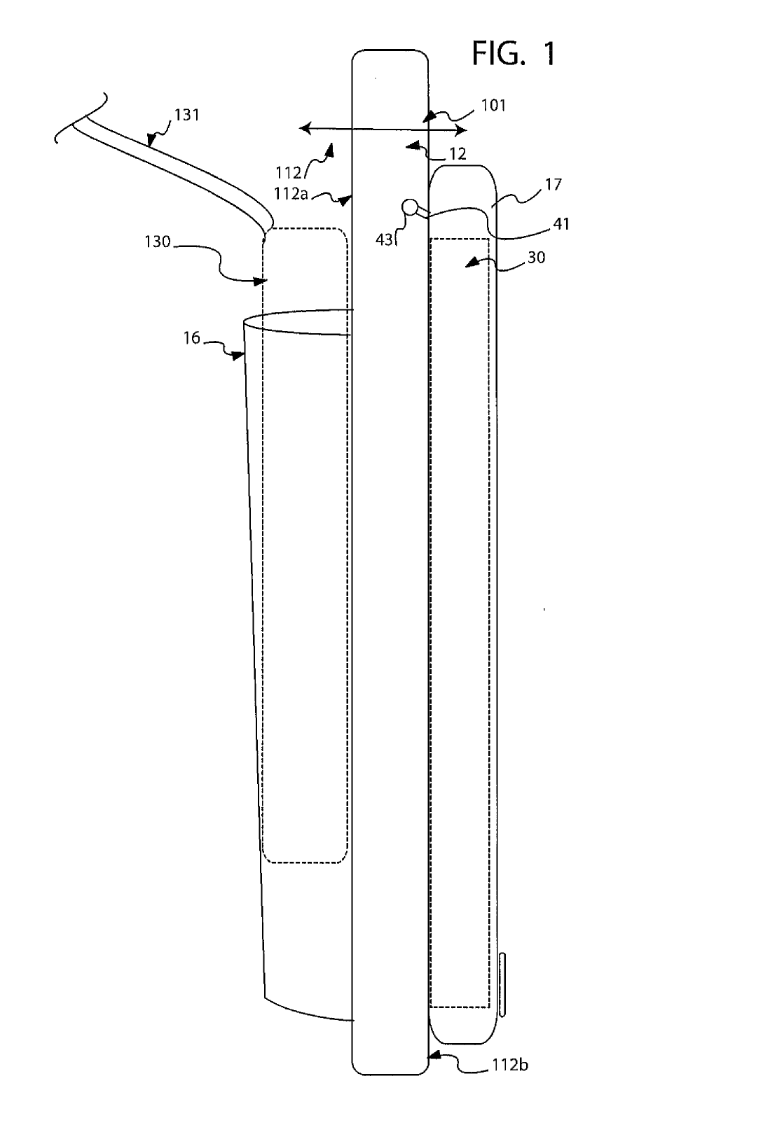

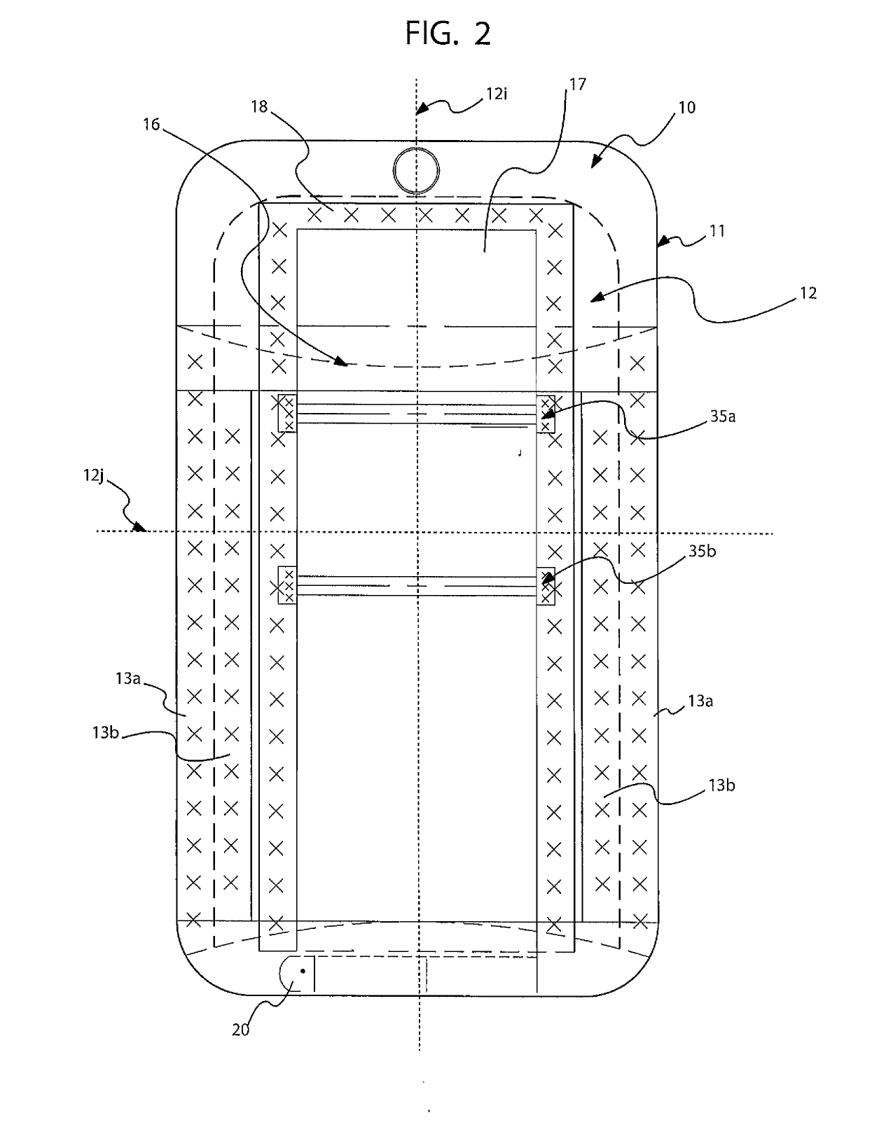

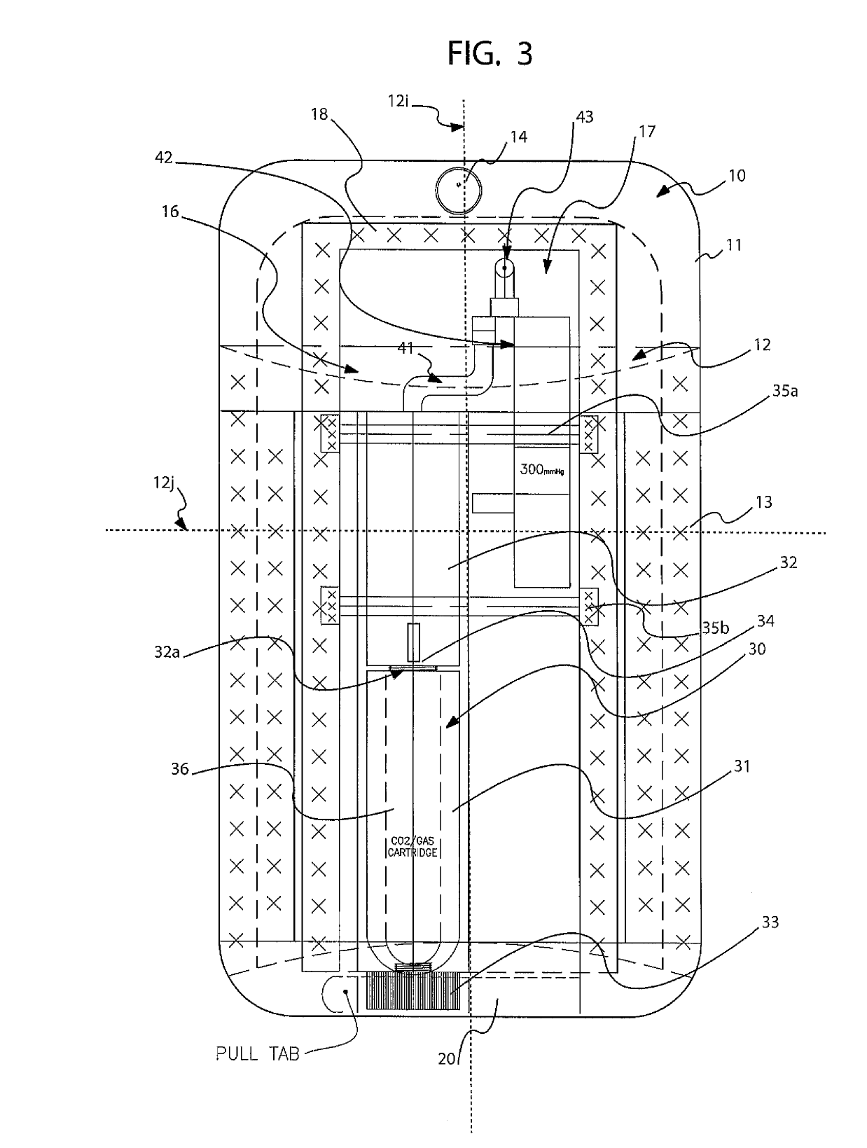

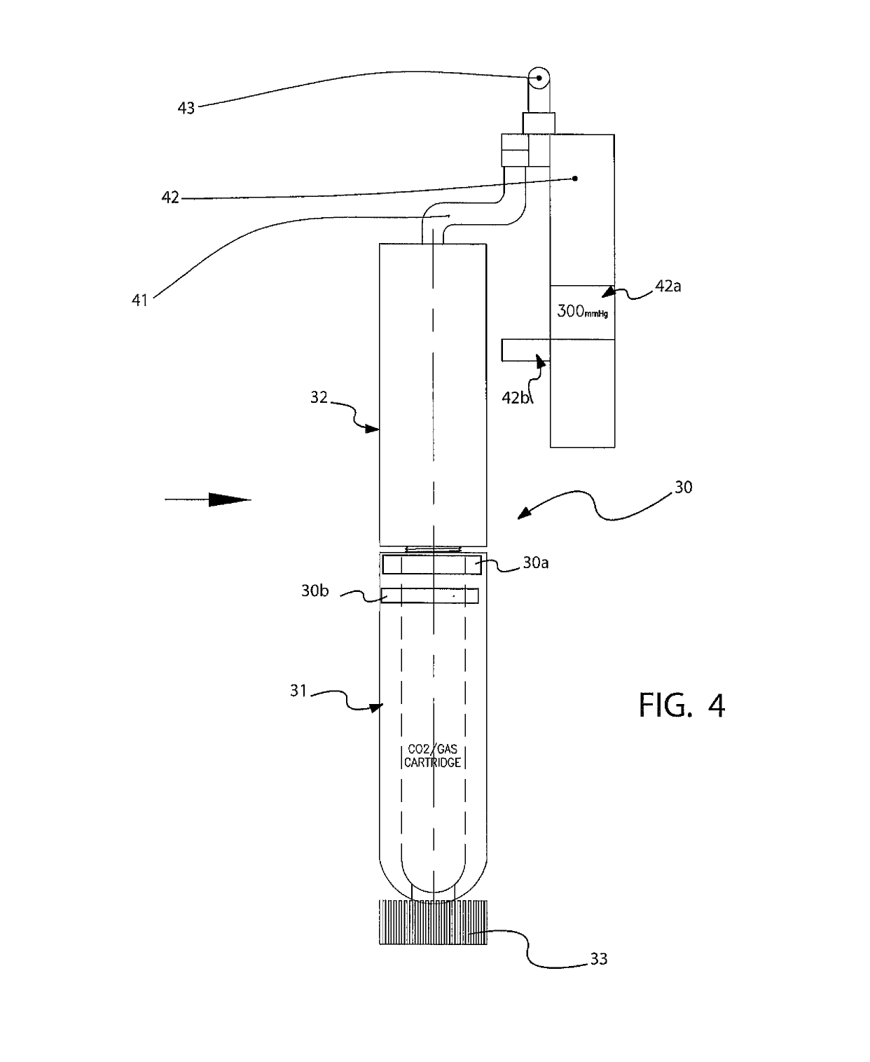

FIG. 1 shows a simplified side view of the embodiment shown in FIGS. 2 and 3. As shown, there is first compartment 12, a second compartment 17 and inflator 30 disposed in second compartment 17. Second compartment 17 is disposed on the second face 112b, which is opposite the first face 112a. First face 112a houses pouch 16. Inflator 30 extends longitudinally along, or substantially parallel to the longitudinal axis 12i (See FIG. 2) of first compartment 12 and has a line 42 that feeds into injection point 43, to feed inflating fluid such as CO2 into first compartment 12. This infusion line can then be coupled to a syringe which can then be inserted into a user to provide the user with a positive pressure infusion system configured to provide fluids to the user.

FIG. 2 is a back view of the device or container 10 shown in FIG. 1 which includes a first compartment 12 having welded edges 13 which forms a sealed compartment 12 forming a bladder which forms a limited volume. Compartment 12 forms a sealed compartment that can be expanded via a fluid from an inflator 30 (See FIG. 1 and FIG. 3). Compartment 12 is sealed by weld lines 13a and 13b This container includes a sleeve 16 which allows an infusion bag such as infusion bag 130 to be inserted therein. Weld line 13a is for securing the outer rim of compartment 12 as well as securing sleeve 12. Weld line 13b is for also securing compartment 12. The infusion bag can be in the form of a liquid filled bag such as a saline or blood filled bag which can be used to insert fluid into a person's body under pressure. This suggested pressure level can be in the form of a preset pressure level such as 300 mm/HG (mercury) of pressure. While that pressure level is indicated, any suitable pressure level can be used. This sleeve 16 can be configured to selectively allow infusion bags to be removed or inserted therein. An additional compartment 17 is formed by another connection of material formed by weld line 18. This connection can be in the form of a welding of additional material over the outside of the first compartment 12 on the side opposite sleeve 16. For example, this welding forms a substantially sealed container 17 which seals the inflator 30 therein to keep the inflator 30 associated with the container and to keep parties from tampering with inflator 30.

The container 10 extends along a longitudinal axis 12i and has a length along this longitudinal axis such that first compartment 12 and second compartment 17 both extend lengthwise along this longitudinal axis. The length of this extension is longer than the width which extends transverse along latitudinal axis 12j. Thus, both the first compartment 12 and the second compartment 17 have lengths that extend parallel or substantially parallel to each other.

While second compartment 17 is substantially closed, it does have a pull tab 20 which allows limited access to inflator 30.

As shown in FIG. 3, pull tab 20 can be pulled away to expose dial 33 which can be used to selectively activate inflator 30 to drive a compressed fluid container or cartridge 36 from a closed condition to an open condition. Inflator 30 is fluidly coupled to first container 12 via a manifold or line 41. Inflator 30 includes a first section 31 and a second section 32. First section 31 is configured to rotate relative to section 32, such that when dial 33 is turned, it rotates first section 31 and drives it via a screw drive or screw connection 32a, towards second housing section 32. This movement drives cartridge 36 towards opener 34, which can be in the form of a spike or open channel for puncturing cartridge 36 and which can include a passage for receiving fluid passed from cartridge 36 into manifold 41. To keep second housing section 32 stable or stationary, there are struts 35a and 35b coupled to or welded to the container 10 which keep second body section 32 from rotating. Housing sections 31 and 32 can be made from a substantially hard plastic shell, such as from a molded plastic. Manifold or line 41 extends from the housing of inflator 30 to an indicator 42, and then out eventually to compartment 12. Indicator 42 can be in the form of a pressure indicator and which is shown in greater detail in FIG. 4.

FIG. 4 is a side view of the inflator 30, the pressure indicator 42 and the manifold 41. Indicator 40 includes an indicator section 42a as well as a bleeder valve 42b. Indicator section 42a can be in the form of any suitable indicator such as a level, a dial, a digital readout or any other type of suitable indicator. Inflator 30 includes a minimum pressure regulator which can include a first pressure regulator 30a, and a second pressure regulator 30b. First pressure regulator 30a is disposed in first housing 31, while second pressure regulator 30b is disposed in second housing 32. This minimum pressure regulator which can be the combination of these two adjustable pressure regulators can be used to set a minimum pressure level for the pressure leaving the inflator housing and entering manifold 41. While inflator 30 can be set or configured to expel a pressurized fluid such as a gas at a minimum pressure level, to insure that the pressure level once exceeding the minimum level is not above a predetermined higher level such as above 330 MM/HG, 300 MM/HG or any other preset level, a bleeder valve 42b with a pre-set release pressure is included. Bleeder valve 42b is coupled to the indicator and disposed downstream of the inflator housing and can be a one-way valve which opens once the pre-set top pressure level is exceeded. Thus, this system with the inflator having a pre-set condition for a minimum base pressure, and a pre-set condition for a maximum top pressure, creates a self-regulating pressurized system. This pressure regulated system can automatically inject additional pressurized fluid such as CO₂ gas to selectively inflate, or at least add pressure to compartment 12 within a pre-set range. As long as the cartridge or pressurized container 31 contains additional pressurizing capacity, then the system can keep being automatically regulated in terms of pressure. In this embodiment, element 43 can be configured as the injection point or the point of entry for manifold 41 into compartment 12.

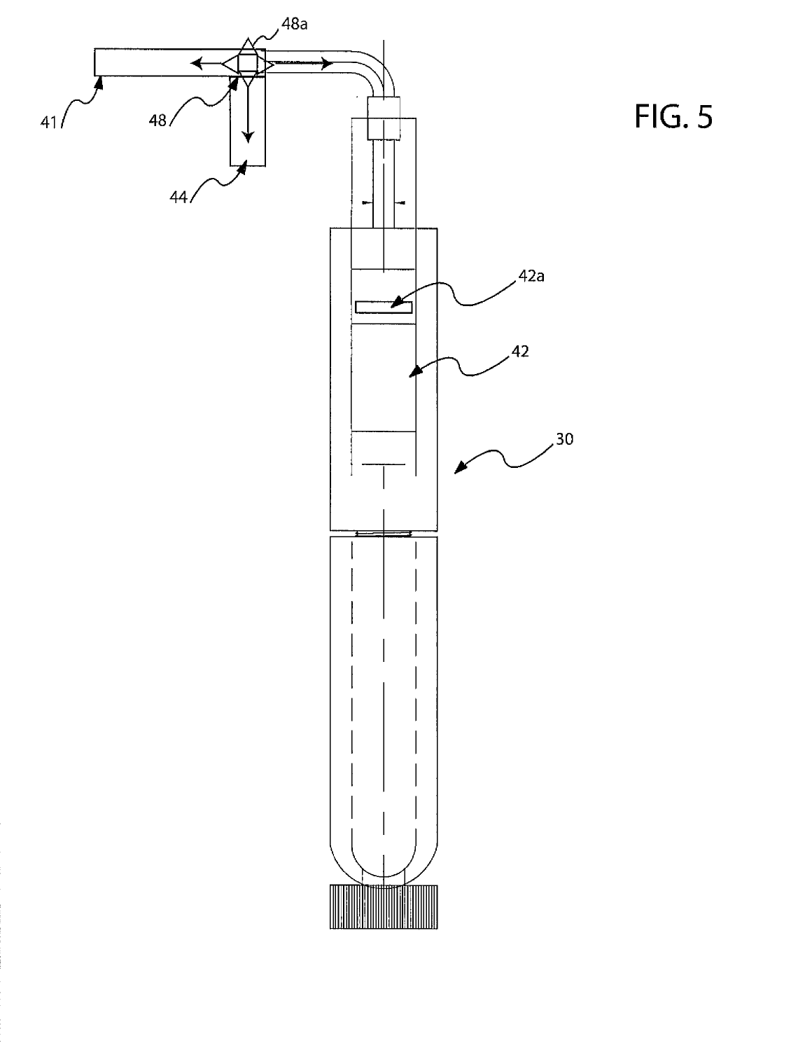

FIG. 5 shows an alternative embodiment, which shows a three-way valve 48 which is coupled along manifold or line 41 and which selectively can be dialed to expel the pressurized gas via a turning of the valve 48. This valve can be set to either allow fluid to pass along manifold 41 without bleeding out from channel 44 or the valve can be opened by turning dial 48a which opens the valve to channel 44 to allow fluid to bleed out of the system.

This embodiment shows inflator 30 also having an indicator section 42a disposed adjacent to the inflator 30. As described above, the indicator 42a is used to indicate a pressure level in the system. Thus this system which includes a bleeder valve 48 can be used in conjunction with indicator 42 which may include a bleeder valve or pop-off valve in the indicator section. Alternatively, three-way valve 48 can also include a pop-off or bleeder valve disposed therein, which can automatically open if a pre-set pressure is exceeded, thereby allowing fluid to drain or escape from the system out channel 44 and preventing compartment 12 from receiving too much pressure. If for example, compartment 12 is under too much pressure, because this compartment is configured to have a limited volume, it would ultimately pop and be rendered useless. Because these components are generally used in a medical setting, the constant popping of inflatable compartments would be disruptive and counterproductive for administering medicaments to a patient. Therefore, both the base line, or minimum pressure is regulated and the maximum pressure is regulated.

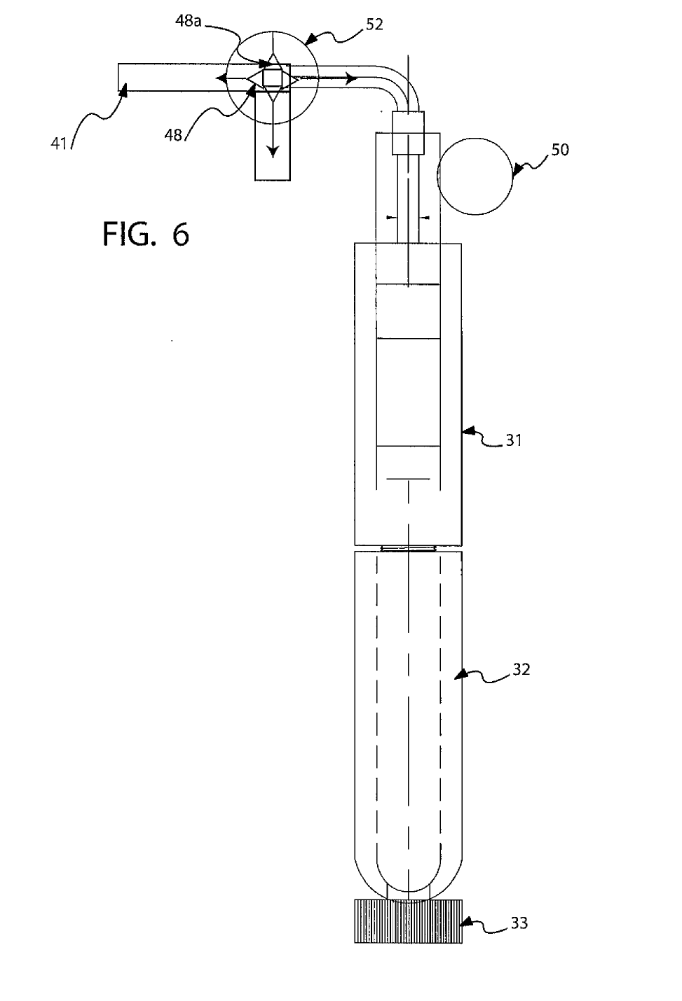

FIG. 6 is a side view of another embodiment. This embodiment includes a three-way valve which includes a dial 48a as well as an indicator or dial controller 52, which controls the rotation of dial 48a as well as provides indication of the type of rotation of dial 48a. For example, this dial controller/indicator 52 can be set to control the rotation of dial 48a in only one direction such as clockwise.

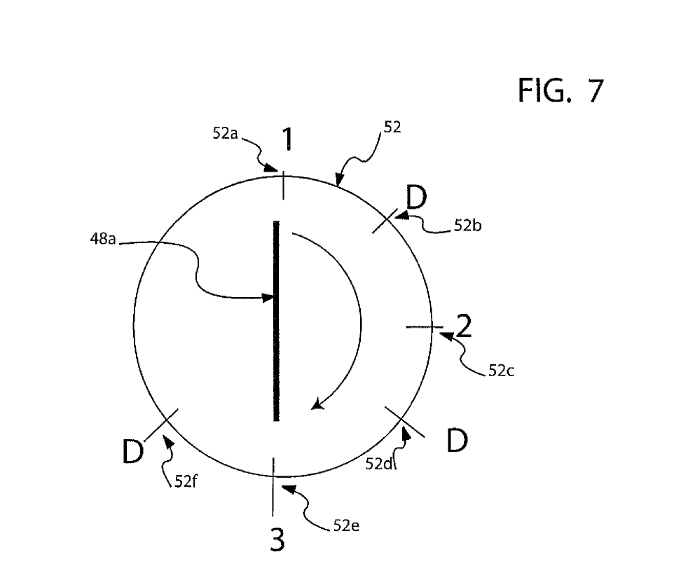

FIG. 7 shows a close up view of this dial 48a and dial controller 52 as well. In this view there is shown a set of indications positioned at a first position 52a, a second position 52b a third position 52c, a fourth position 52d, a fifth position 52e and a sixth position 52f. This dial control 52 can be used to control the rotation of dial 48a, such that it can only rotate in one direction such as shown in this drawing as clockwise. In addition, the dial controller 52 can be used to control the dial such that once it is rotated from a first position such as from position 52a, to second position 52b it cannot be rotated back.

First position 52a indicates a position where the manifold is in a closed position allowing a first infusion bag inserted into sleeve 16 to be acted on. Next inflator 30 pressurizes the system. When that infusion bag is used up, dial 48a can be rotated to the second position 52b which is a deflate position. Once the compartment 12 is deflated, an additional infusion bag can be inserted into sleeve 16. Then, dial 48a can be rotated to the next position 52c, thereby inflating compartment 12 again to thereby provide pressure on the section infusion bag. When this infusion bag is used up, dial 48a, which can only be rotated in one direction such as clockwise, can be rotated towards position 52d to deflate the compartment 12 again, thereby allowing the infusion bag to be removed from sleeve 16. Next, when a third infusion bag is inserted into sleeve 16, dial 48a can be rotated to position 52e whereby compartment 12 can be inflated again thereby putting pressure on the third infusion bag. Once this infusion bag has drained, dial 48 can be rotated again to another position 52f to allow for final deflation. This preset dial controller 52 can be used to automatically control how infusion bags are used with this inflator 30, and to automatically count how many bags are used with a particular inflator 30. While six (6) positions are shown, multiple additional positions can be used as well if necessary.

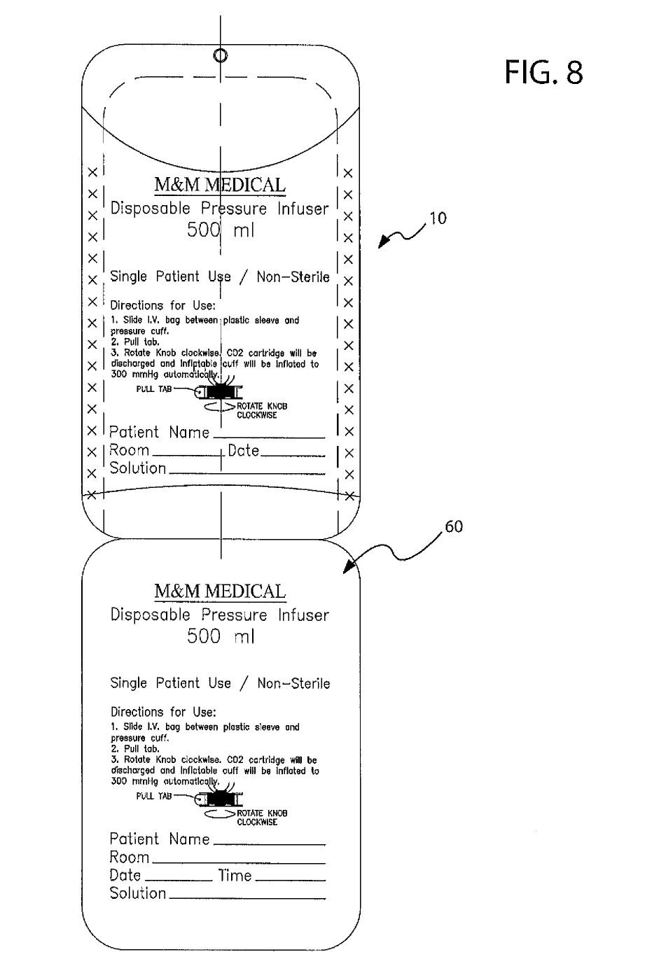

FIG. 8 is an additional view of an infusion bag which includes an additional tag coupled to the container 10. This tag 60 is configured to allow a person such as a medical professional to follow particular directions as well as to provide indication of the patient, the room, the date, the time and the solution used for infusion.

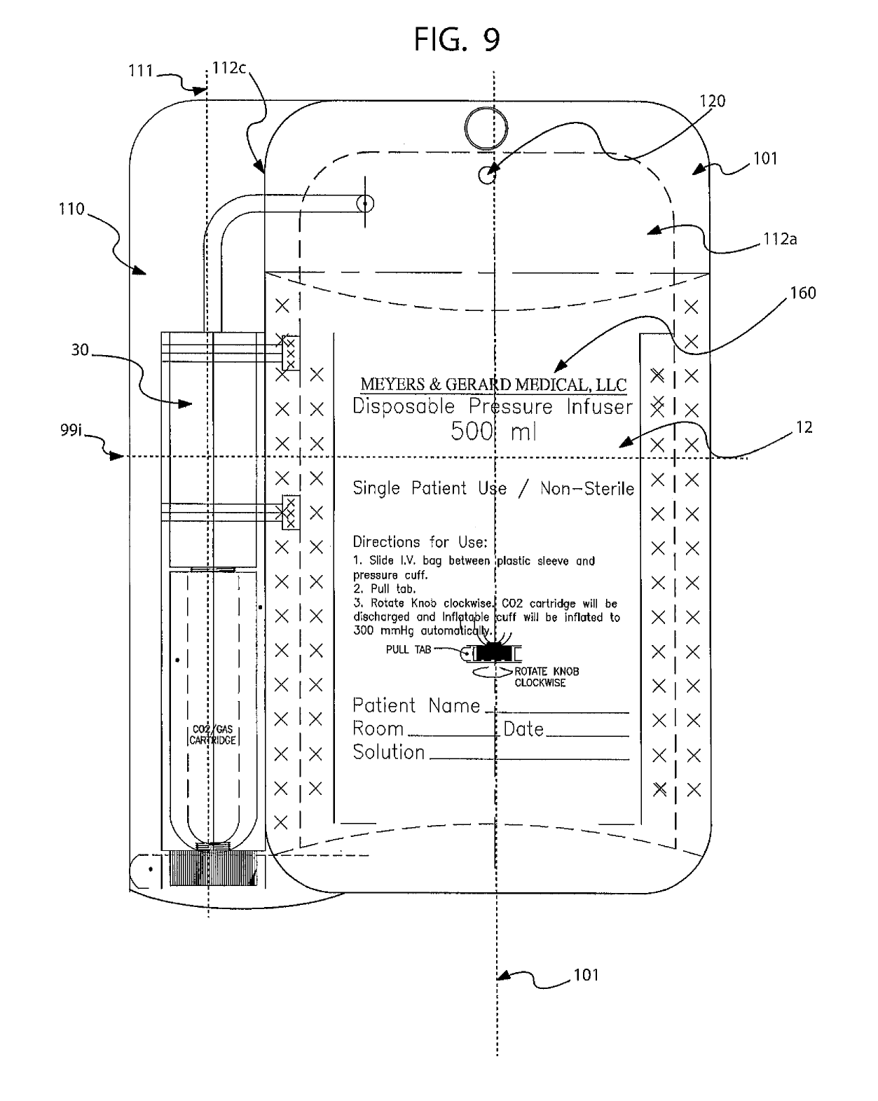

FIG. 9 is another embodiment of the container system. This view shows a back view of a container 101 which has all of the same elements of container 10 except with this system, the pouch or container 110 for the inflator 30 is disposed on the side of the bag, and not in a central region of the bag. The container extends along a longitudinal axis 101i and the inflator container 110 also extends along a longitudinal axis 111 which is substantially parallel to the longitudinal axis 101 of the extent or length of the container 110. The length or longitudinal axis is the dimension that is longer than the latitudinal axis 99i or width of the device. There is also a pressure relief valve 120 which is set to relieve the inner chamber of the device once it is inflated by the inflator 30. The pressure relief valve is set to relieve pressure at 411 mm/Hg or 8 psi. In this way, the inflator 30 which inflates the bag cannot inflate the bag to a pressure that is higher than the bag can hold. The positioning of this pressure relief valve reduces the need for an in-line pressure relief valve in the system. However, an in-line pressure relief valve which is in-line with line 41 is also possible. As shown, line 41 feeds into injection point 43 to feed fluid such as CO2 into the bag.

With the pressure relief valve 120 incorporated into the bag, the bag would not be in danger of bursting when it is being inflated. This view shows the back face 112b as well as side 112c which allows inflator 30 to be coupled therein via second container 110.

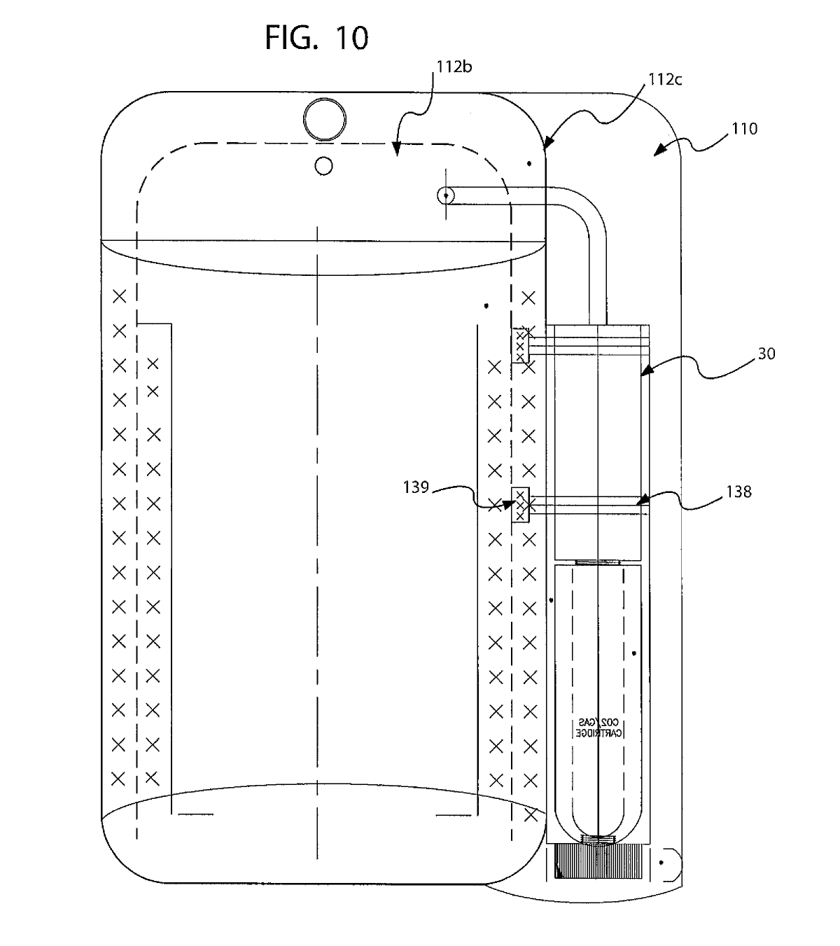

FIG. 10 shows a front view of the bag as well which shows the front side 112a of container 12 as well as the side portion 112c of the container 12. This view shows that the inflator 30 is secured by straps 138 which are bonded via bonds 139 to compartment 110

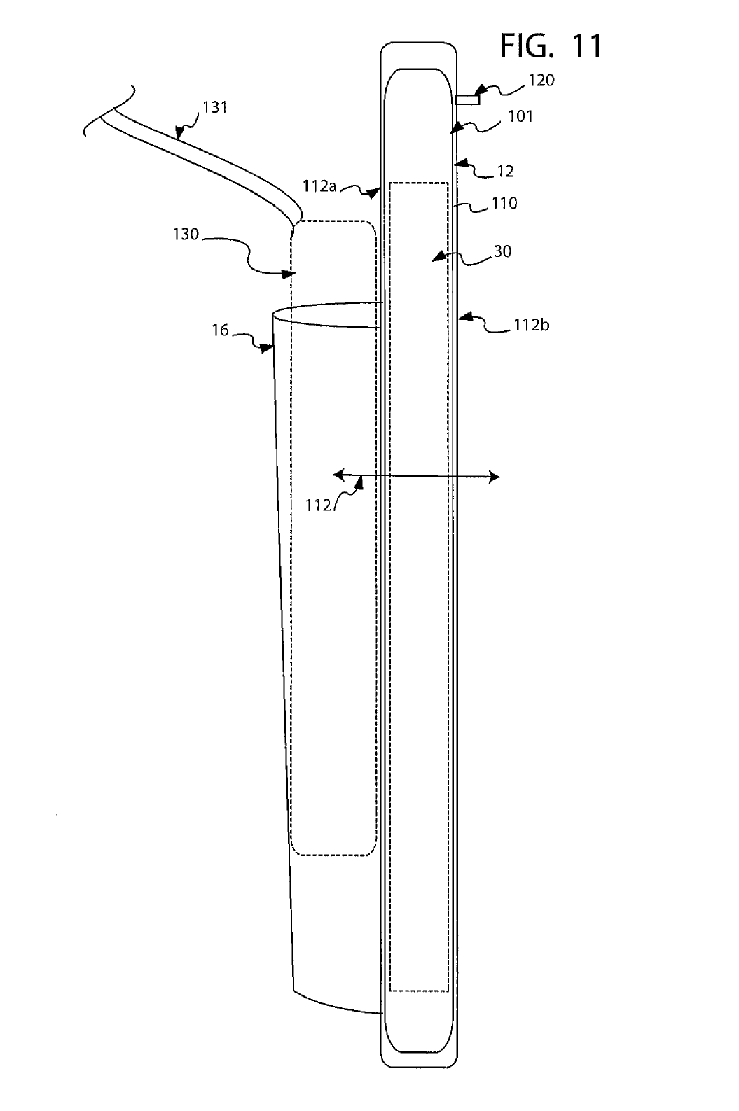

FIG. 11 is a side view of the device showing sealed container 12 which can be used with either the embodiment 10 or 101 and which is used to inflate to put pressure on infusion bag 130 which can contain a solution of treatment material such as saline or blood. There is shown second container 16 which is used to hold the infusion bag 130 such that wherein when container 12 expands via the injection of fluid from inflator 30, then this container expands laterally as shown by arrow 112 to put pressure on infusion bag 130 to cause fluid to flow out of infusion bag 130 and into infusion line 131. First compartment 12 which comprises a bladder has a front face 112a, a back face 112b and a side 112c, with inflator 30 coupled to the side 112c of container 12. This side is laterally offset from container 12.

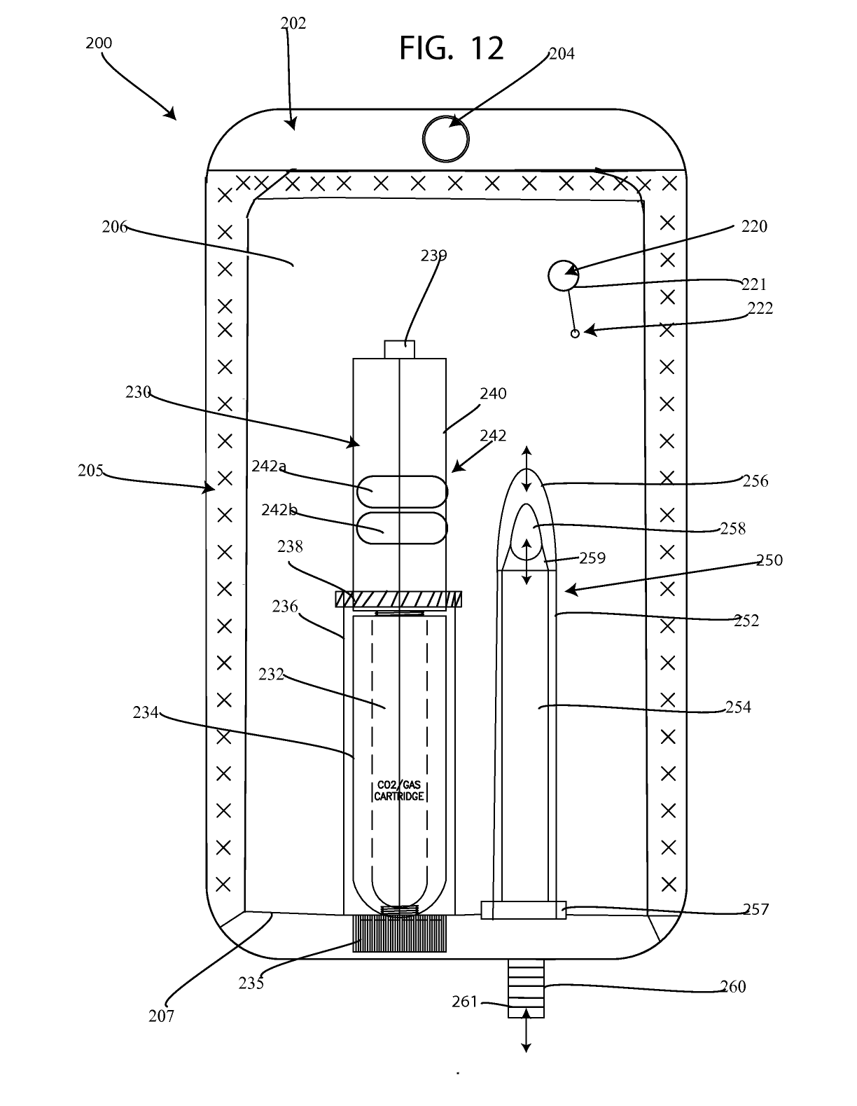

FIG. 12 is a side view of another embodiment. In this embodiment 200, there is disclosed a bag 202 which has a hole 204 which is an opening for hanging the bag on a hook. The bag includes welded edges 205 and 207 which form an enclosed inner inflatable region 206 which allows the bag to be inflated by an inflator 230. There is also a gauge 250 which measures the pressure inside of the bag.

Bag 202 is configured to house a substantial portion of inflator 230 as well as gauge 250. In addition there is a port 220 having a removable cover 221 which includes a tail 222 which allows a user to pull on the tail 222 to remove cover 221 to open the port. The opening of the port allows for the bag to be deflated thereby allowing for the re-use of the bag. The bag is configured to receive an infusion bag on at least one side such that once the bag is inflated, it applies pressure on the infusion bag as is known from the disclosure above. Thus, on at least one side of the bag is a pocket for receiving an infusion bag which may be filled with blood, saline or other liquids for distribution into a patient.

Inflator 230 is powered by any suitable inflation element but in this embodiment is a cartridge 232 which can be a selectively removable cartridge. This selectively removable cartridge 232 is disposed inside of a housing 234, which is disposed inside of a container 236. Coupled to an end of housing 234 is a cap 235 which is a screw on cap and selectively removable allowing for removal of the end and selective removal of cartridge 232. In addition as cap 235 is turned it pushes cartridge 232 into a puncture element to thereby open cartridge 234 and its gas contents into container 240.

Coupled to housing 234 is a valve container 240 containing at least one valve 242. The at least one valve 242 can include multiple valves 242a and 242b wherein these valves can be used to control the amount of pressure provided inside of the bag 202, such as inside of inflatable region 206. With two valves in use these valves are coupled in parallel or in series to receive the pressure inside of container 240 on one end and then distribute the gasses at the opposite end such as towards 239 at a different pressure. Thus, these valves are fluidly coupled to the open portion of the container 240 which receives fluid from cartridge 234. The fluid must then flow through at least one of the valves where it is regulated in pressure and then distributed out from port 239 at a desired pressure. This pressure can be for example 300 MM/HG. Thus, these valves allow for only a controlled release from this container into inflatable region 206 via port 239 which forms a fluid channel into inflatable region 206.

A pressure indicator 250 is disposed inside of bag 202, such that this pressure indicator 250 includes an outer seal 252, an inner casing body 254, an end cover 256, an inner nipple 258 coupled to one end 259 and a sliding indicator 260 having indicia 261 indicating the pressure level inside of inflatable region 206 Inner casing body is coupled to the bag 202 via a secure closed connection such as a weld 257. When pressure inside the inflation region 206 increases, this places increasing pressure on end cover 256, driving nipple 258 inside of inner casing 254, and thereby applying pressure on sliding indicator 260. A soldering 257 is coupled to the bottom of the inner casing 254 and the inflatable bag 206 to secure this pressure indicator 250 inside.

Thus, the inflator 230 and the pressure indicator 250 are sealed or at least substantially sealed inside of inflatable region 206 with only sealed openings for a cap 235, and for sliding indicator 260. Thus, this design can be used to self-inflate with the turning of cap 235 driving pressurized container or cartridge 232 to open thereby allowing fluid or gas such as CO2 to enter into the valve container 240, wherein the pressure leaving cartridge 232 is regulated before it enters into inflatable region 206 which is essentially a sealed bag. This allows for expansion of inflatable region 206 in a manner shown as well in FIG. 11 thereby allowing pressure to be applied to an infusion bag disposed in a pouch such as pouch 16 shown in FIG. 11, and causing sliding indicator 260 to extend out from pressure indicator 250.

Thus, the designs disclosed above provide a simple, self-contained, self-inflating infusion bag which is compact and easy to operate.

Accordingly, while at least one embodiment of the present invention have been shown and described, it is to be understood that many changes and modifications may be made thereunto without departing from the spirit and scope of the invention as defined in the appended claims.

What is claimed is:

1. A container comprising:
a) a first compartment;
b) a sleeve configured to receive at least one container for holding fluid;
c) a second compartment coupled to said first compartment, outside of said first compartment;
d) at least one inflator configured to pump a fluid into said first compartment wherein said at least one inflator is disposed sealed within said second compartment; and
e) at least one fluid channel coupled to said first compartment and to said at least one inflator for allowing fluid to flow from said at least one inflator to said first compartment.

2. The container as claimed in claim 1, wherein said first compartment is a bladder with a substantially finite volume and is substantially sealed such that fluid is expressed into and from said first compartment substantially through said at least one fluid channel.

3. The container as claimed in claim 1, further comprising at least one valve for selectively allowing fluid to flow into and out of said at least one compartment.

4. The container as in claimed in claim 3, wherein said at least one valve is at least a three-way valve.

5. The container as claimed in claim 4, wherein said three-way valve comprises a valve for allowing fluid to pass from said inflator to said at least one compartment and at least one bleeder valve for allowing fluid to be expressed from at least one of said first compartment and said inflator.

6. The container claimed in claim 5, wherein said three-way valve is configured to allow fluid to be expressed from said first compartment while preventing fluid from being expressed from said inflator.

7. The container as claimed in claim 1, wherein said at least one inflator comprises a pressurized cartridge.

8. The container as claimed in claim 7, wherein said pressurized cartridge comprises a propellant filled cartridge housing at least one fluid under pressure.

9. The container as claimed in claim 8, wherein said pressurized cartridge comprises a pressurized CO₂ cartridge having at least one section for selectively opening said pressurized cartridge.

10. The container claimed in claim 1, wherein said at least one second container is open to receive said at least one fluid port but is substantially sealed.

11. The container as claimed in claim 1, wherein said at least one first compartment is open to receive said at least one fluid port but is substantially sealed.

12. The container as claimed in claim 1, further comprising at least one pressure gauge.

13. The container as claimed in claim 1, wherein said at least one pressure gauge is coupled in line with said at least one fluid channel.

14. The container as claimed in claim 1, wherein said inflator comprises at least one pressurized container, at least one housing, and at least one actuator configured to selectively actuate said inflator.

15. The container as claimed in claim 15, wherein said actuator comprises a drive and at least one opener, wherein said drive is configured to drive said pressurized container into said opener to selectively open said at least one pressurized container.

16. The container as claimed in claim 15, wherein said at least one housing for said inflator comprises at least two parts made from rigid plastic, wherein said first compartment extends along a longitudinal axis and said second compartment extends along a longitudinal axis wherein said longitudinal axis of said first compartment is substantially parallel with said longitudinal axis of said second compartment.

17. The container as claimed in claim 1, further comprising at least one pressure regulator disposed between said at least one first compartment and said at least one second compartment, wherein said at least one pressure regulator is coupled to said least one first part of said housing.

18. The container as claimed in claim 17, wherein said at least one first part of said housing is movable relative to said at least one second part of said housing to adjust a pressure on said at least one pressure regulator.

19. The container as in claim 1, wherein said first compartment has a front face and a back face and at least one side, wherein said first compartment extends along a longitudinal axis and wherein said second compartment has a longitudinal axis that is coaxial with said longitudinal axis of said first compartment, wherein said second compartment is coupled to said at least one side of said first compartment.

20. The container as in claim 1, further comprising a pressure relief valve coupled to said first compartment and configured to relieve pressure inside of said first compartment.
